(19) United States
(12) Patent Application Publication
(10) Pub. No.: US 2026/0328951 A1
(43) Pub. Date: Oct. 22, 2026

(54) MICROCHIP DEVICE TO MONITOR A PERSONS WHEREABOUTS

(71) Applicant: Edward Lee Pemberton, Bothell, WA (US)

(72) Inventor: Edward Lee Pemberton, Bothell, WA (US)

(21) Appl. No.: 19/655,723

(22) Filed: Apr. 23, 2026

Related U.S. Application Data
(60) Provisional application No. 63/790,740, filed on Apr. 18, 2025.

(51) Int. Cl. G01S 19/16; A44C 25/00; A61B 5/00; G01S 19/34; G01S 19/36; G08B 21/02; G08B 25/10

(52) U.S. Cl. CPC G01S 19/16; A44C 25/001; A61B 5/6861; G01S 19/34; G01S 19/36; G08B 21/0269; G08B 21/0288; G08B 25/10

(57) ABSTRACT

A concealed or implanted tracking device. The tracking device may have its location accessed by a monitoring center where a lost or abducted person's responsible parent or guardian may initiate monitoring so that help may be sent to rescue the abductee or lost person.

### TECHNICAL FIELD

This description relates generally to devices that may be concealed on or in the body to monitor the location of a person.

### BACKGROUND

Children who are kidnaped are typically difficult to locate, a they may not know where they are if the get away, and of course the kidnapper is trying to conceal them. Accordingly, a device that is not easily separated from a person, or known to be carried by a person, that generates a location signal may be desirable to help a child be located before the location device may be discovered and removed by a kidnapper.

In the case of the elderly or infirm who may wander away and become lost locating them may be problematic. A panic button, a cell phone or the like may easily be separated from the person and they may not be able to be located by conventional methods. Accordingly in these cased it may be desirable to provide a locating or tracking device so that they may be located and assisted.

### SUMMARY

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

The present example provides a system for monitoring a person's location via a typically concealed device so that in case of abduction or loss the person may be recovered.

Many of the attendant features will be more readily appreciated as the same becomes better understood by reference to the following detailed description considered in connection with the accompanying drawings.

### DESCRIPTION OF THE DRAWINGS

The present description will be better understood from the following detailed description read in light of the accompanying drawings, wherein:

FIG. 1 shows a device for monitoring a person's location using a concealed tracking device.

FIG. 2 shows a system for monitoring a person's location.

FIG. 3 shows an alternative system for monitoring a person's location.

Like reference numerals are used to designate like parts in the accompanying drawings.

### DETAILED DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

The examples below describe a device to continually monitor the location of a person. Although the present examples are described and illustrated herein as being implemented in an implanted device, the system described is provided as an example and not a limitation. As those skilled in the art will appreciate, the present examples are suitable for application in a variety of different types of implants or wearable systems including those worn such as jewelry, piercings and the like.

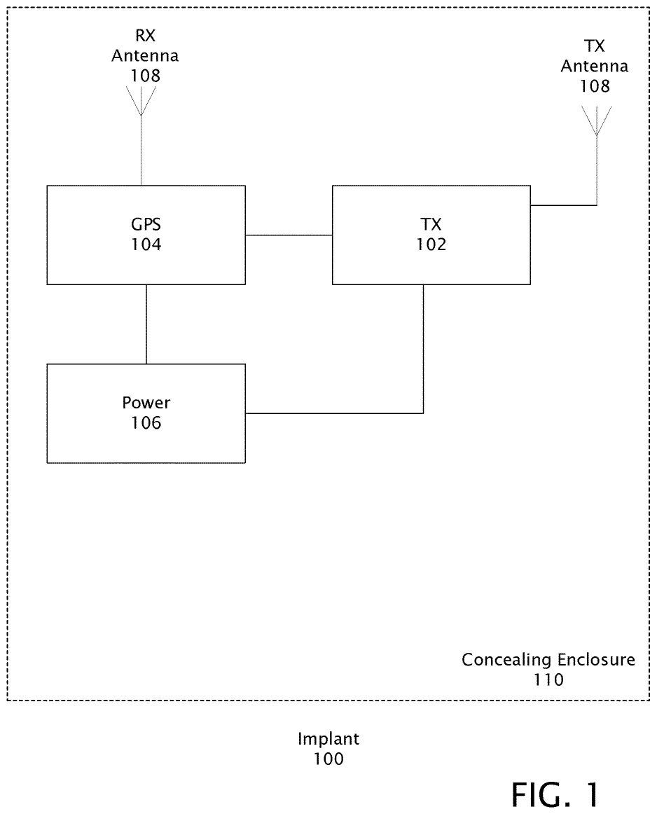

FIG. 1 shows a general system diagram of a device 100 for monitoring a person's location using a concealed tracking device.

A microchip device is typically encased 110 and implanted in a person to monitor whereabouts at all times mitigating issues of kidnapping/trafficking/lost or displaced children/dementia and critical care individuals. Alternatively the microchip may be concealed in a piece of jewelry or piercing. Implanting is typically done in a location that is not easily seen, and in a manner such that the microchip may not easily be removed if found. Likewise when mad into jewelry or piercings care is taken to hide the presence of the tracking device.

The size of the microchip 100 is typically no larger than 0.125″×0.125″×0.03125″ thick and may include versions that include a maximum thickness of 0.0625″. The microchip typically has GPS or equivalent capabilities as well as wireless capabilities such as satellite, cellular, or the like.

The microchip, or equivalently the microchip locator module, may include a typical transmitter 102 coupled to a locator module 104 capable of receiving a location signal such as GPS or the like. The GPS location coordinates are then submitted to the transmitter 103 for relay to a monitoring center. The GPS locator 104 may be included on the microchip or may be electronically coupled by Bluetooth pairing or the like, to another device worn by the person such as a GPS watch, fitness device or the like. In the case of the GPS module being present in the microchip continuous, or intermittent, reporting of the location may be sent to the monitoring center. In the case of a GPS device being paired to the microchip the last known position may be sent to the monitoring center once coupling is lost.

Power may be provided by an included power cell 106 with a long life. Alternatively a rechargeable power cell may be provided that is typically recharged by electronic coupling by a charger placed in close proximity to the power cell. Such a device would typically include a charge storage device and the recharging/coupling circuitry. Alternatively, a passive microchip may be provided that receives power to activate transmission of coordinates from some outside source emitting electronic signals that may be recognized by the microchip and converted to power to operate the device.

In further alternative examples of a power source 106 a photovoltaic cell may be included to convert light into energy to power the microchip. This is especially useful in jewelry and piercing devices with exposed surfaces. If under the skin some power may be absorbed by a photocell if it is implanted close to the surface of the skin.

A transmitting antenna 108 is typically provided to transmit the location signal. The antenna may be disposed on the microchip, or may extend from it. In the case of jewelry or piercings the antenna may be molded or formed into the body of the piece. For example in the case of a necklace and pendant, the antenna could be concealed in the chain, and the electronics of the microchip in the pendant.

A receiving antenna 112 for taking in a GPS signal may be configured in similar fashion to the transmitting antenna 108.

The microchip may be implanted under the skin in a place that may not be easily seen. Alternatively, even if the device may be seen it typically may not be easily removed. Such an implanted device may be coated with a nontoxic and non-irritating material so that it does not cause undue discomfort to the wearer.

In the case of a jewelry or piercing item, the microchip may be concealed 110 in the jewelry or piercing item, preferably in a location that may not have conducting material interfering with the electronic signal being transmitted.

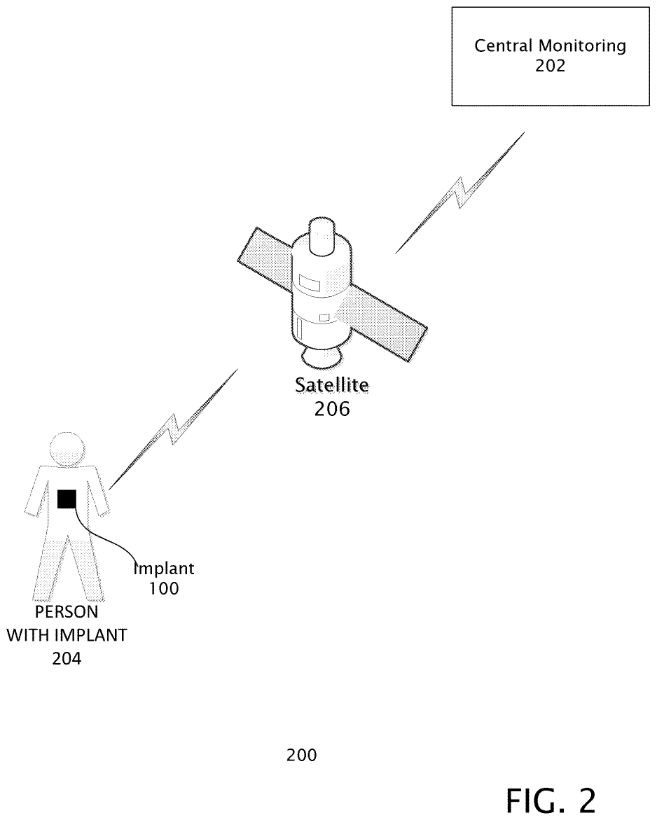

FIG. 2 shows a system for performing monitoring of the implant 200. The device 100, worn by a person 204 may be monitored at a central location 202 that provides the constant whereabouts of the individual at all times. Such monitoring may be critical to saving lives.

The monitoring may be via satellite 206 or the like. Monitoring may be continuous, activated remotely, or activated by the wearer. The microchip typically has GPS, or equivalent, capabilities with the location coordinates of the microchip being transmitted wirelessly to a monitoring center, where assistance may be dispatched to the location and the wearer of the microchip.

A satellite 206 may be coupled to the device 100, and the monitoring location 202 to provide connectivity.

The monitoring station 202 may typically be set up to provide location information to a subscriber such as family, emergency personnel or the like. Location information may include map coordinates, coordinates superimposed on a map or other additional information to aid in finding the person.

Satellite 206 or equivalent, including a network of satellites, may be provided to couple the microchip 100 to the monitoring station 202.

A person with an implant 204 may be coupled to a satellite, or satellite network 206. Satellite 204 is in turn coupled to and in communication with central monitoring 202. Implant 100 may be constructed as previously described.

The GPS, or equivalent systems for providing a location signal are conventionally constructed and is not shown.

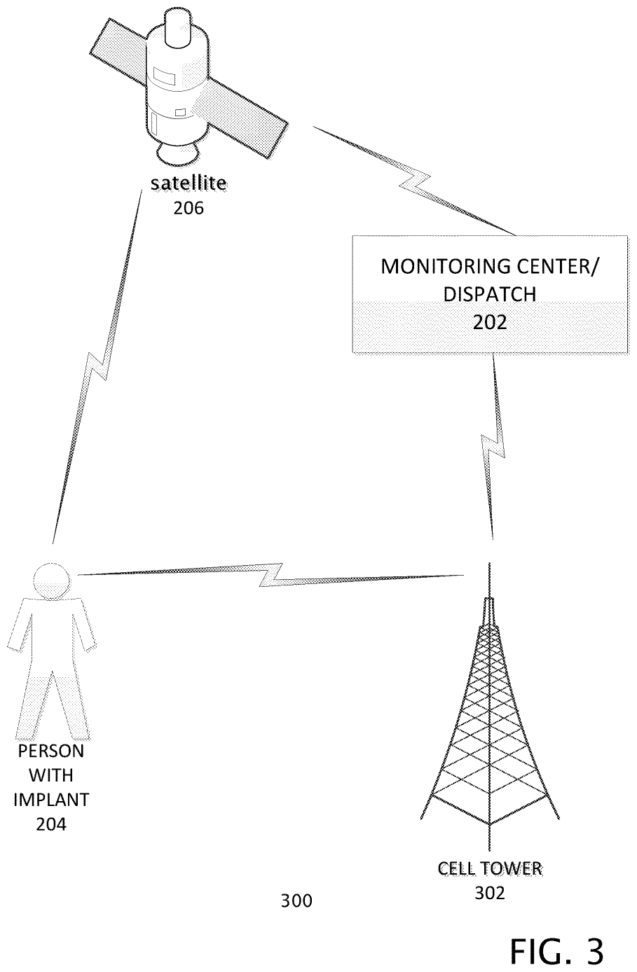

FIG. 3 shows an alternative monitoring system. The monitoring may be via satellite 206 or the like. In this example a cell tower 302 may be utilized in addition to or instead of the satellite 206 to provide a communications medium. In all examples monitoring may be continuous, activated remotely, or activated by the wearer. The microchip typically has GPS capabilities with the location coordinates of the microchip being transmitted wirelessly to a monitoring center, where assistance may be dispatched to the location and the wearer of the microchip.

A person with an implant 204 may be coupled to a satellite 206 and/or a cellular network represented by cell tower 302. Monitoring center 202 may be coupled to satellite 206 and/or cellular network represented by cellular tower 302.

The GPS, or equivalent systems for providing a location signal are conventionally constructed and is not shown.

1. A system to monitor a person's whereabouts comprising:
an implantable microchip tracking device including:
a transmitter;
a transmitting antenna coupled to the transmitter;
a GPS receiver sending a location signal to the transmitter;
a receiving antenna receiving a location signal; and
a power source coupled to the transmitter;
a control center continuously receiving the person's whereabouts; and
at least one satellite of a plurality of satellites electronically coupling the implantable microchip tracking device to the control center

2. The system to monitor a person's whereabouts of claim 1, in which the implantable microchip tracking device is encased in an inert material.

3. The system to monitor a person's whereabouts of claim 2, in which the implantable microchip tracking device is implanted in the body of a wearer.

4. The system to monitor a person's whereabouts of claim 1, in which the implantable microchip tracking device is concealed in jewelry.

5. The system to monitor a person's whereabouts of claim 4, in which the jewelry is a necklace with a pendant and a chain.

6. The system to monitor a person's whereabouts of claim 5, in which the necklace includes a transmitting antenna.

7. The system to monitor a person's whereabouts of claim 5, in which the pendant includes the transmitter.

8. The system to monitor a person's whereabouts of claim 4, in which the jewelry is a bracelet.

9. The system to monitor a person's whereabouts of claim 1, in which the implantable microchip tracking device is concealed in a piercing.

10. An implantable microchip tracking device including:
a transmitter;
a transmitting antenna coupled to the transmitter;
a GPS receiver sending a location signal to the transmitter;
a receiving antenna receiving a location signal; and
a power source coupled to the transmitter;

11. The implantable microchip tracking device of claim 10, in which the implantable microchip tracking device is encased in an inert material.

12. The implantable microchip tracking device of claim 11, in which the implantable microchip tracking device is implanted in the body of a wearer.

13. The implantable microchip tracking device of claim 10, in which the implantable microchip tracking device is concealed in jewelry.

14. The implantable microchip tracking device of claim 13, in which the jewelry is a necklace with a pendant and a chain.

15. The implantable microchip tracking device of claim 4, in which the necklace includes a transmitting antenna.

16. The implantable microchip tracking device of claim 14, in which the pendant includes the transmitter.

17. The implantable microchip tracking device of claim 13, in which the jewelry is a bracelet.

18. The system to monitor a person's whereabouts of claim 10, in which the implantable microchip tracking device is concealed in a piercing.
